(19) United States
(12) Patent Application Publication
(10) Pub. No.: US 2016/0296254 A1
(43) Pub. Date: Oct. 13, 2016

(54) SYSTEM AND METHOD FOR PEDICULAR AND LAMINAR SPINAL FIXATION

(71) Applicant: John R. Dimar, II, Anchorage, KY (US)

(72) Inventor: John R. Dimar, II, Anchorage, KY (US)

(21) Appl. No.: 15/091,136

(22) Filed: Apr. 5, 2016

Related U.S. Application Data
(60) Provisional application No. 62/143,887, filed on Apr. 7, 2015.

(51) Int. Cl. A61B 17/70

(52) U.S. Cl. CPC A61B 17/7001; A61B 2017/567

(57) ABSTRACT

A system and method for providing fixation to a spine according to one example embodiment includes a hook portion with a plurality of planes of adjustment and an attachment portion connected to the hook portion. The attachment portion is configured to connect to the top of a pedicle screw to provide pedicular and laminar fixation to the spine at a vertebra to which the pedicle screw is connected.

### CROSS-REFERENCE TO RELATED APPLICATIONS

This application claims priority to U.S. Provisional Application No. 62/143,887, entitled “System and Method for Pedicular and Laminar Spinal Fixation” and filed on Apr. 7, 2015, which is incorporated by reference herein in its entirety.

### BACKGROUND

1. Field of the Disclosure

The present invention relates to systems and methods for pedicular and laminar spinal fixation.

2. Description of the Related Art

Currently, pedicular spinal fixation systems and laminar spinal fixation systems are used independently to provide fixation and stability to the spine to treat spinal deformities, fractures, tumors and numerous degenerative conditions. The spinal fixation systems typically use surgically placed pedicle screws to attach rods to the spine. After the rods and screws have been surgically placed, however, the pedicle screws often break or pull out of the spine. When the screws break and pull out of the spine, spinal fixation can be lost, resulting in catastrophic failure and severe complications. Moreover, current spinal fixation systems usually require the removal of rod and pedicle screw lock-up or attachment mechanisms to place supplemental fixation, a difficult and time consuming process. Further, current spinal fixation systems do not provide both pedicular and laminar fixation at a single vertebra because of space constraints.

Accordingly, it will be appreciated that systems and methods for pedicular and laminar spinal fixation that are easy to apply and that prevent pedicle screws from failing and pulling out of the spine are desired.

### SUMMARY

A system for providing fixation to a spine according to one example embodiment includes a hook portion having a plurality of planes of adjustment and an attachment portion connected to the hook portion. The attachment portion is configured to connect to the top of a pedicle screw to provide pedicular and laminar fixation to the spine at a vertebra to which the pedicle screw is connected.

A system for providing fixation to a spine according to another example embodiment includes a hook portion having a plurality of planes of adjustment and an attachment portion connected to the hook portion. The attachment portion connects to the top of a previously surgically placed pedicle screw to provide pedicular and laminar fixation to the spine at a vertebra to which the pedicle screw is connected.

A system for providing fixation to a spine according to another example embodiment includes a hook portion having a plurality of planes of adjustment and an attachment portion connected to the hook portion. The attachment portion connects to the top of a pedicle screw, and the hook portion connects to a vertebra to which the pedicle screw is connected.

### BRIEF DESCRIPTION OF THE DRAWINGS

The above-mentioned and other features and advantages of the various embodiments, and the manner of attaining them, will become more apparent and will be better understood by reference to the accompanying drawings.

FIG. 1 is a perspective view of a system for pedicular and laminar spinal fixation according to multiple embodiments and alternatives;

FIG. 2 is a perspective view of the system for pedicular and laminar spinal fixation connected to a pedicle screw according to multiple embodiments and alternatives;

FIG. 3a is a top plan view of the system for pedicular and laminar spinal fixation with an attachment portion with a single attachment aperture according to multiple embodiments and alternatives;

FIG. 3b is a top plan view of the system for pedicular and laminar spinal fixation with the attachment portion with multiple attachment apertures according to multiple embodiments and alternatives;

FIG. 3c is a top plan view of the system for pedicular and laminar spinal fixation with the attachment portion with slotted attachment apertures according to multiple embodiments and alternatives;

FIG. 4 is a perspective view of the attachment portion with the pedicle screw and a plug with a top threaded extension according to multiple embodiments and alternatives;

FIG. 5a is a perspective view of a placement screw for the system for pedicular and laminar spinal fixation according to multiple embodiments and alternatives;

FIG. 5b is a perspective view of a plug for the system for pedicular and laminar spinal fixation according to multiple embodiments and alternatives;

FIG. 6 is a cross sectional view of the attachment portion with the pedicle screw, the plug and the placement screw according to multiple embodiments and alternatives;

FIG. 7a is a perspective view of the system for pedicular and laminar spinal fixation according to multiple embodiments and alternatives;

FIG. 7b is a perspective view of the system for pedicular and laminar spinal fixation according to multiple embodiments and alternatives;

FIG. 8 is a perspective view of the system for pedicular and laminar spinal fixation according to multiple embodiments and alternatives;

FIG. 9 is a perspective view of the system for pedicular and laminar spinal fixation according to multiple embodiments and alternatives;

FIG. 10 is a perspective view of the system for pedicular and laminar spinal fixation according to multiple embodiments and alternatives; and

FIG. 11 is a perspective view of the system for pedicular and laminar spinal fixation according to multiple embodiments and alternatives.

### DETAILED DESCRIPTION

The following description and drawings illustrate embodiments sufficiently to enable those skilled in the art to practice the present invention. It is to be understood that the disclosure is not limited to the details of construction and the arrangement of components set forth in the following description or illustrated in the drawings. The invention is capable of other embodiments and of being practiced or of being carried out in various ways. For example, other embodiments may incorporate structural, chronological, electrical, electronic, process and other changes. Examples merely typify possible variations. Individual components and functions are optional unless explicitly required, and the sequence of operations may vary. Portions and features of some embodiments may be included in or substituted for those of others. The following description is, therefore, not to be taken in a limited sense.

Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings and mountings. In addition, the terms “connected” and “coupled” and variations thereof are not restricted to physical or mechanical connections or couplings.

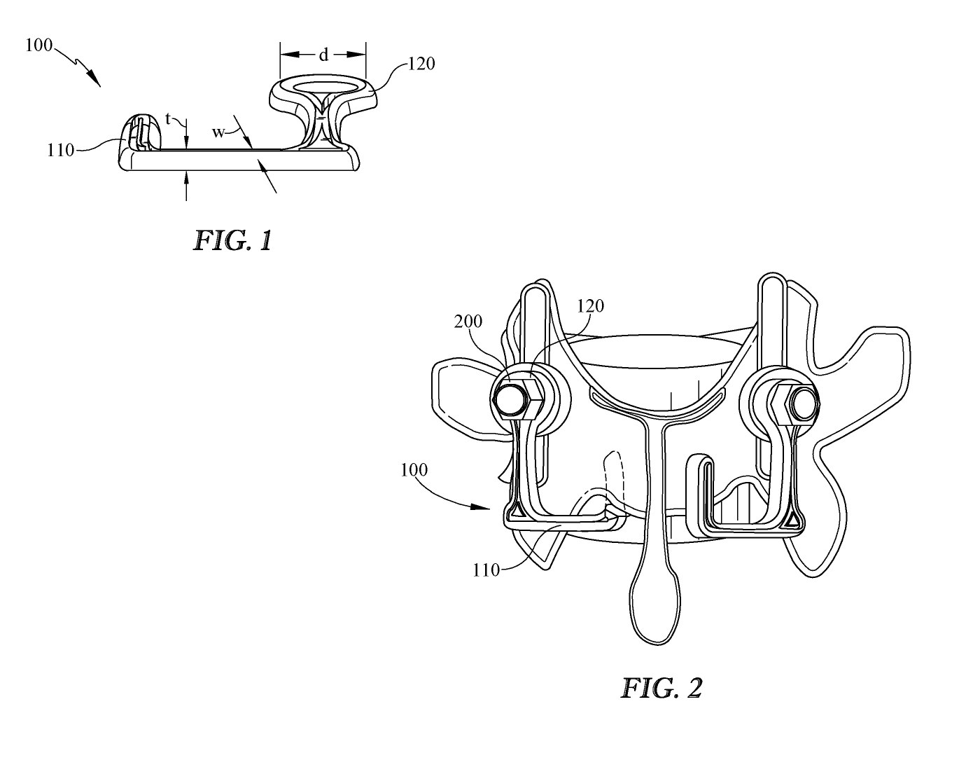

Turning now to the drawings, and more particularly to FIG. 1, an example embodiment of a system for pedicular and laminar spinal fixation 100 is illustrated. The system 100 includes a hook portion 110 with a plurality of planes of adjustment and an attachment portion 120 connected to the hook portion. As shown in FIG. 2, the attachment portion 120 connects to the top of a pedicle screw 200 to provide pedicular and laminar fixation to the spine at a vertebra to which the pedicle screw is connected.

With reference to FIG. 1, the hook portion 110 may have a variety of foot widths w, foot thicknesses t, throat diameters d and angulations or bends to accommodate various laminar shapes and sizes and to accommodate placements at various vertebrae portions, such as sublaminar placements, supra laminar placements and transverse process placements, for example. In some embodiments, the hook portion 110 may accommodate placements in a 360° arc extending around the center axis of the yoke of the pedicle screw to attach to various vertebrae portions to fortify the pedicular fixation and to prevent screw pullout and failure.

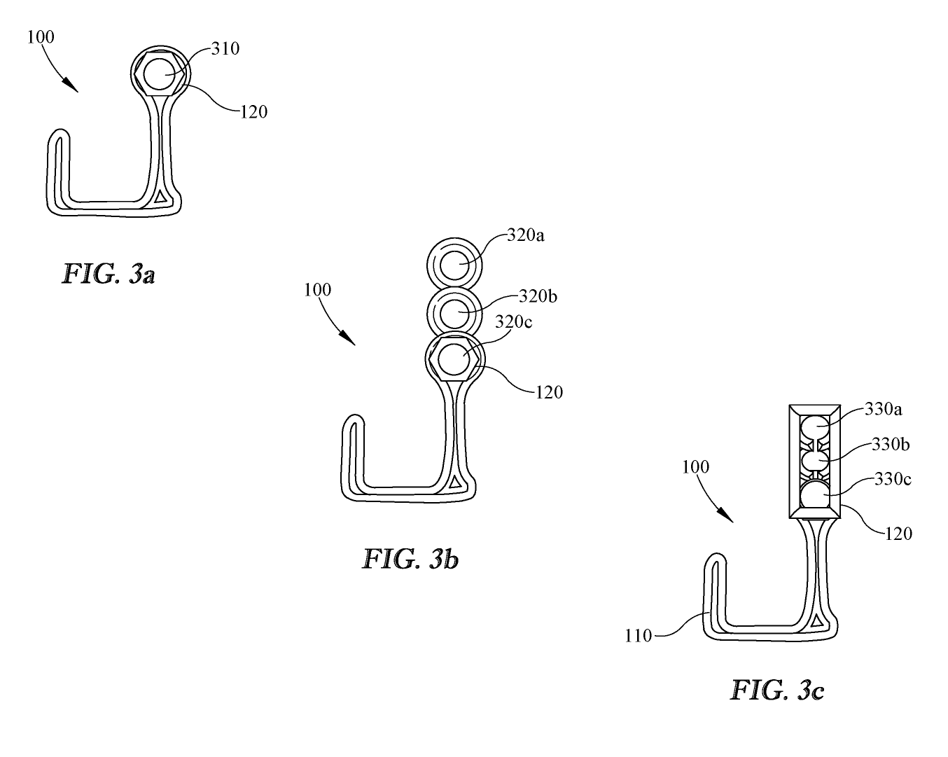

As illustrated in FIG. 3a, in some embodiments, the attachment portion 120 of the system 100 may include a single attachment aperture 310. Turning to FIG. 3b, in some embodiments, the attachment portion 120 may include multiple attachment apertures 320a, 320b and 320c. As shown in FIG. 3c, in some embodiments, the attachment portion 120 may include slotted attachment apertures 330a, 330b and 330c. In certain embodiments, the slotted attachment apertures 330a, 330b and 330c allow the hook portion 110 to be adjusted cephalad and caudally.

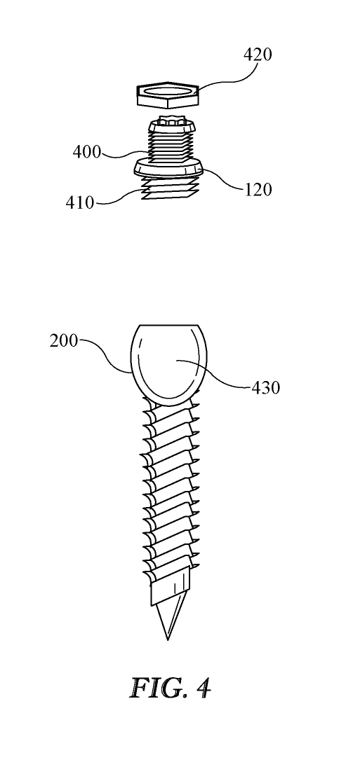

In some embodiments, the attachment portion 120 may connect to the top of the pedicle screw 200 through the use of a plug 410 with a top threaded extension 400 as illustrated in FIG. 4. In certain embodiments, the plug 410 may be ridged. In certain embodiments, a nut 420 may also be used to connect the attachment portion 120 to the top of the pedicle screw 200.

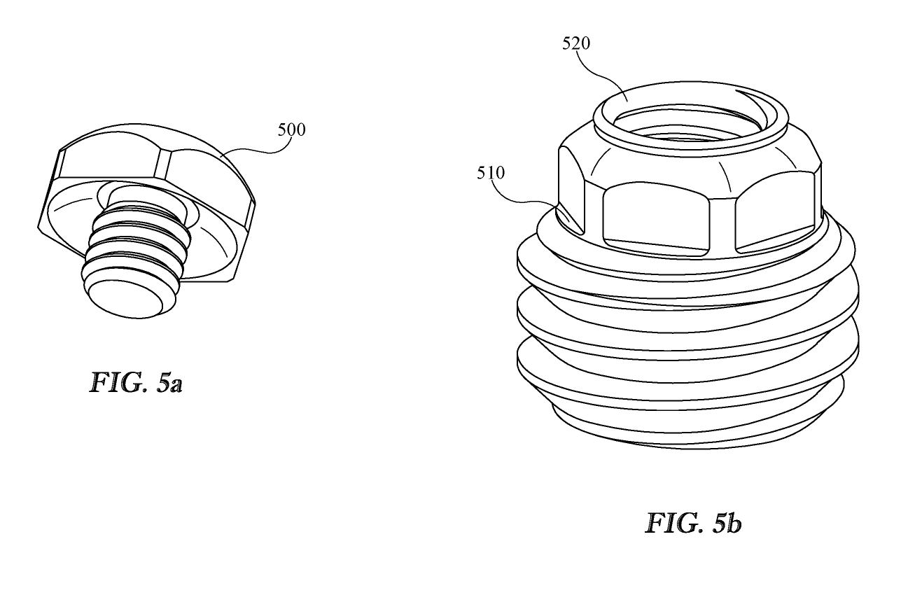

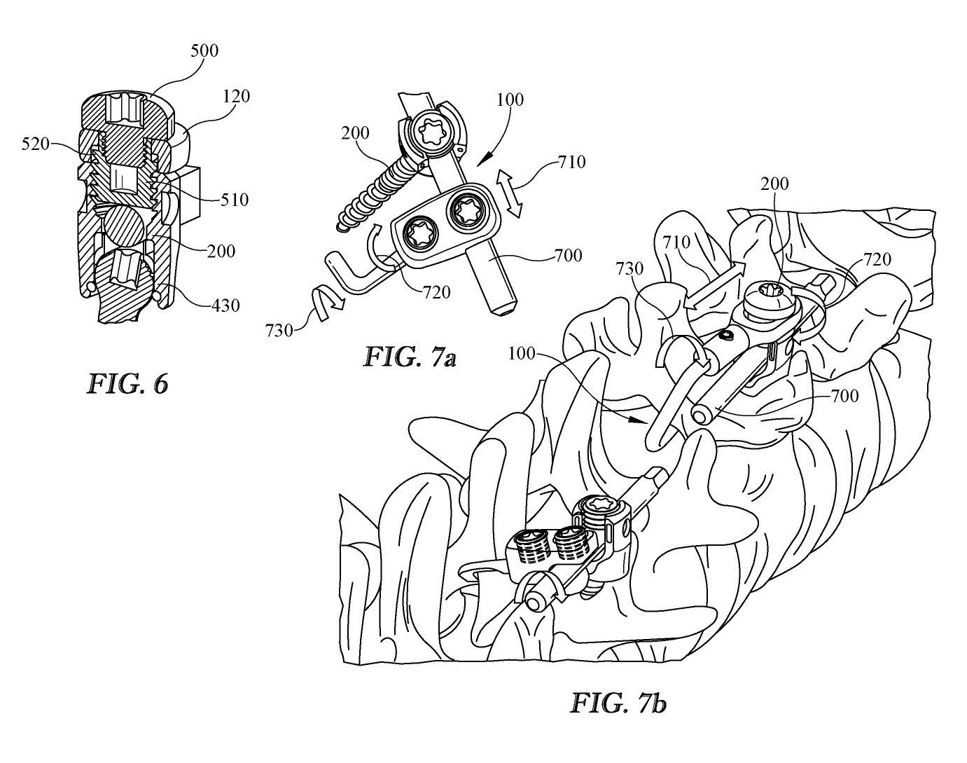

In some embodiments, the attachment portion 120 may connect to the top of the pedicle screw 200 through the use of a plug 510 with a female receptacle 520 and a placement screw 500 as illustrated in FIGS. 5a, 5b and 6. In certain embodiments, the plug 510 with the female receptacle 520 may be ridged. In certain embodiments, a nut may also be used to connect the attachment portion 120 to the top of the pedicle screw 200.

Returning to FIG. 1, in certain embodiments, the system for pedicular and laminar spinal fixation 100 does not require any additional intra-screw head rod space and has a low profile. Thus, the system 100 is compact and easy to surgically place and remove.

The components of the system for pedicular and laminar spinal fixation 100 may be made of a variety of sturdy biocompatible materials, such as titanium, stainless steel or cobalt-chrome alloys, for example. In some embodiments, the system 100 may be integrally formed with the pedicle screw. In other embodiments, the system 100 may be distinct from the pedicle screw.

In use, in multiple embodiments, the system 100 attaches to the top of an existing, previously surgically placed pedicle screw to provide supplemental fixation. In some embodiments, the system 100 may allow screws with weak fixation, such as screws in osteoporotic bone, for example, to be salvaged before they break or pull out of the spine. In some embodiments, the system 100 also prevents the removal or modification of major existing hardware, such as rods or pedicle screws, for example. Thus, the system 100 increases pedicle screw pullout fixation strength in a practical and easy to apply manner.

In some embodiments, the system 100 attaches to the top of a pedicle screw when the pedicle screw is surgically placed to provide supplemental fixation.

In some embodiments, the attachment portion 120 of the system 100 connects to the top of the pedicle screw 200 through the use of the plug 410 with the top threaded extension 400 as illustrated in FIG. 4. In certain embodiments, the plug 410 with the top threaded extension 400 is inserted into a yoke 430 of the pedicle screw 200 and the attachment portion 120 is positioned around the top threaded extension 400 with the top threaded extension 400 in the attachment aperture of the attachment portion 120. In certain embodiments, the nut 420 may be placed around the top threaded extension 400 on top of the attachment portion 120. In certain embodiments, the positioning of the attachment portion 120 around the top threaded extension 400 of the plug 410, which is inserted into the yoke 430 of the screw 200, decreases the footprint of the vertebra fixation, decreases the bulk and size of the fixation and allows the fixation to fit onto the spine in an improved manner.

In some embodiments, the attachment portion 120 connects to the top of the pedicle screw 200 through the use of the plug 510 with the female receptacle 520 and the placement screw 500 as illustrated in FIGS. 5a, 5b and 6. In certain embodiments, the plug 510 with the female receptacle 520 is inserted into the yoke 430 of the pedicle screw 200, the attachment portion 120 is positioned around the plug 510 with the plug 510 in the attachment aperture of the attachment portion 120 and the placement screw 500 is inserted into the female receptacle 520 of the plug 510. In certain embodiments, a nut may be placed around the placement screw on top of the attachment portion 120. In certain embodiments, the positioning of the attachment portion 120 around the plug 510 and insertion of the placement screw 500 into the female receptacle 520 of the plug 510, which is inserted into the yoke 430 of the screw 200, decreases the footprint of the vertebra fixation, decreases the bulk and size of the fixation and allows the fixation to fit onto the spine in an improved manner.

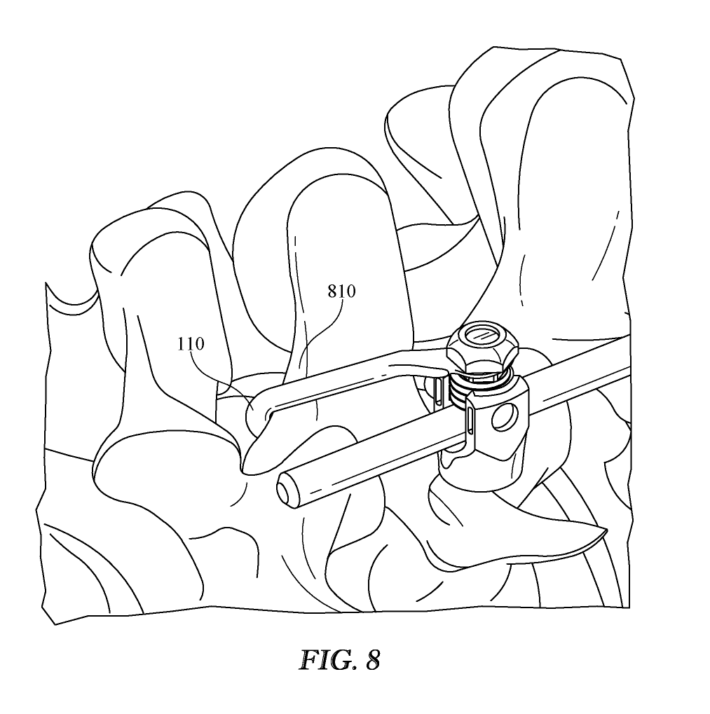

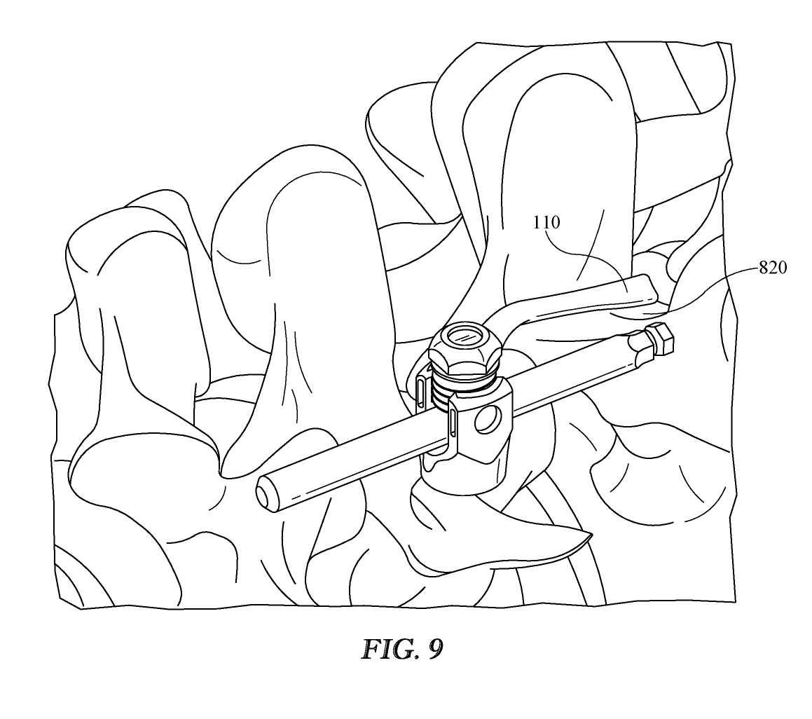

As shown in FIGS. 7a and 7b, in some embodiments, the system for pedicular and laminar spinal fixation 100 connects to a spinal rod 700 to bolster the vertebra fixation.

As illustrated in FIGS. 7a and 7b, the hook portion of the system 100 may be adjusted in a plurality of planes of adjustment. In some embodiments, the hook portion may be adjusted cephalad and caudally 710. In some embodiments, the hook portion may be adjusted rotationally 720 around the head of the pedicle screw for coronal placement. In some embodiments, the hook portion may be adjusted sagittally 730 utilizing the head of the pedicle screw to adjust it to fit over or under a lamina. In some embodiments, the hook portion may be adjusted in the sagittal, axial and coronal planes in a 360° arc extending around the top of the pedicle screw.

The system 100 may be placed at a variety of locations on the vertebra, such as sublaminar, supra laminar and transverse process, for example. In some embodiments, the hook portion of the system 100 may be placed within a 360° arc extending around the center axis of the yoke of the pedicle screw 200 to attach to various vertebrae portions to fortify the pedicular fixation and to prevent screw pullout and failure.

As shown in FIG. 2, in some embodiments, the hook portion 110 of the system 100 connects to the spine at a vertebra to which the pedicle screw 200 is connected. In other embodiments, the hook portion 110 connects to the spine at another vertebra, for example at a vertebra adjacent to the vertebra to which the pedicle screw is connected.

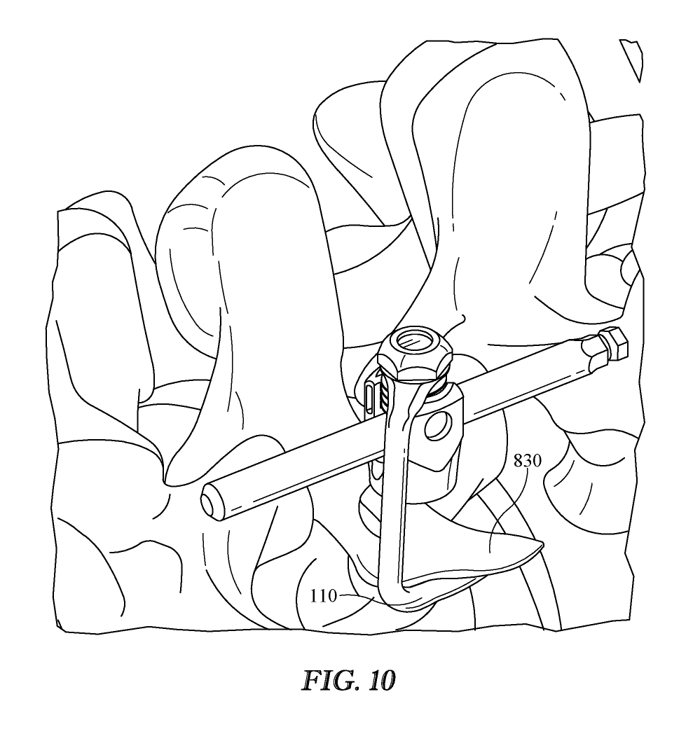

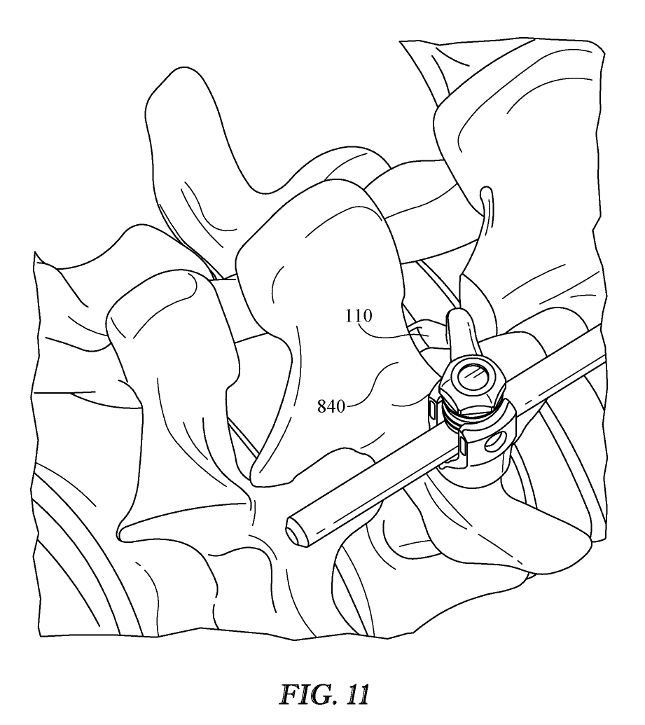

As shown in FIG. 8, in some embodiments, the hook portion 110 is placed on the caudal lamina 810. In some embodiments, the hook portion 110 is placed on the cephalad lamina of an adjacent vertebra 820 as shown in FIG. 9. In some embodiments, the hook portion 110 is placed on the transverse process 830 as shown in FIG. 10. In some embodiments, the hook portion 110 is placed on the cephalad lamina 840 as shown in FIG. 11 to enhance the cephalad extent of the fixation and to decrease the incidence of proximal junctional failure.

In certain embodiments, the system 100 may provide fixation at the cervical-thoracic junction, a difficult area to place pedicle screws and hooks. In certain embodiments, the system 100 may be used for spondylolysis repair by compressing and reducing the laminar pars defect toward the pedicle screw by placing the hook portion caudad under the lamina.

It will be appreciated that the system for pedicular and laminar spinal fixation 100 prevents pedicle screws from failing and pulling out of the spine and increases vertebral fixation strength in an easy to apply manner. The system 100 also increases the adherence of spinal implants to the spine, prevents the dislodgement of spinal implants from the spine and prevents the loss of spinal stabilization.

The foregoing description of several embodiments has been presented for purposes of illustration. It is not intended to be exhaustive or to limit the application to the precise forms disclosed, and obviously many modifications and variations are possible in light of the above teaching. It is understood that the invention may be practiced in ways other than as specifically set forth herein without departing from the scope of the invention. It is intended that the scope of the application be defined by the claims appended hereto.

What is claimed is:

1. A system for providing fixation to a spine, comprising:
a hook portion having a plurality of planes of adjustment; and
an attachment portion connected to the hook portion,
wherein the attachment portion is configured to connect to the top of a pedicle screw to provide pedicular and laminar fixation to the spine at a vertebra to which the pedicle screw is connected.

2. The system of claim 1, wherein the attachment portion has a plurality of attachment apertures.

3. The system of claim 1, wherein the attachment portion is configured to connect to the top of the pedicle screw with a plug having a top threaded extension.

4. The system of claim 1, wherein the attachment portion is configured to connect to the top of the pedicle screw with a plug having a female receptacle and a placement screw.

5. The system of claim 1, wherein the hook portion is configured to be located sublaminar on the vertebra.

6. The system of claim 1, wherein the hook portion is configured to be located supra laminar on the vertebra.

7. The system of claim 1, wherein the hook portion is configured to be located on the transverse process of the vertebra.

8. The system of claim 1, wherein the hook portion may be adjusted cephalad and caudally, rotationally around the head of the pedicle screw for coronal placement and sagittally utilizing the head of the pedicle screw to fit over or under a lamina.

9. A system for providing fixation to a spine, comprising:
a hook portion having a plurality of planes of adjustment; and
an attachment portion connected to the hook portion,
wherein the attachment portion connects to the top of a previously surgically placed pedicle screw to provide pedicular and laminar fixation to the spine at a vertebra to which the pedicle screw is connected.

10. The system of claim 9, wherein the attachment portion has a plurality of attachment apertures.

11. The system of claim 9, wherein the hook portion is located sublaminar on the vertebra.

12. The system of claim 9, wherein the hook portion is located supra laminar on the vertebra.

13. The system of claim 9, wherein the hook portion is located on the transverse process of the vertebra.

14. The system of claim 9, wherein the hook portion may be adjusted cephalad and caudally, rotationally around the head of the pedicle screw for coronal placement and sagittally utilizing the head of the pedicle screw to fit over or under a lamina.

15. A system for providing fixation to a spine, comprising:
a hook portion having a plurality of planes of adjustment; and
an attachment portion connected to the hook portion,
wherein the attachment portion connects to the top of a pedicle screw and wherein the hook portion connects to a vertebra to which the pedicle screw is connected.

16. The system of claim 15, wherein the pedicle screw is a previously surgically placed pedicle screw.

17. The system of claim 15, wherein the hook portion is located sublaminar on the vertebra.

18. The system of claim 15, wherein the hook portion is located supra laminar on the vertebra.

19. The system of claim 15, wherein the hook portion is located on the transverse process of the vertebra.

20. The system of claim 15, wherein the hook portion may be adjusted cephalad and caudally, rotationally around the head of the pedicle screw for coronal placement and sagittally utilizing the head of the pedicle screw to fit over or under a lamina.
